(19) United States
(12) Plant Patent Application Publication
(10) Pub. No.: US 2007/0079407 P1
(43) Pub. Date: Apr. 5, 2007

(54) Strawberry plant named 'Driscoll Bonaire'

(76) Inventor: Kristie L. Gilford, Dover, FL (US)

Correspondence Address: JONES DAY, 222 EAST 41ST ST, NEW YORK, NY, 10017, US

(21) Appl. No.: 11/244,118

(22) Filed: Oct. 4, 2005

(51) Int. Cl. A01H 5/00

(52) U.S. Cl. PLT/209

(57) ABSTRACT

This invention relates to a new and distinct variety of strawberry named ‘Driscoll Bonaire’. The variety is similar to the varieties ‘Driscoll Malibu’ and ‘Driscoll Osceola.’ The variety is distinguished from ‘Driscoll Malibu’ and ‘Driscoll Osceola,’ in particular, by its longer petiole length and wider petiole diameter, longer sepal length and greater sepal width, longer fruit length, greater berry weight and longer harvest interval.

### LATIN NAME OF THE GENUS AND SPECIES OF THE PLANT CLAIMED

The variety is botanically identified as Fragaria×ananassa.

### VARIETY DENOMINATION

The strawberry variety denomination is ‘Driscoll Bonaire’.

### BACKGROUND OF THE INVENTION

The new variety originated as a result of a controlled cross between the strawberry plants ‘Driscoll Marathon’ (U.S. Plant Pat. No. 12,817) and ‘Driscoll Madeira’ (U.S. Plant Pat. No. 14,109) in an ongoing breeding program, and was discovered as a seedling in Hillsborough County, Florida in December 2000. The original seedling of the new cultivar was asexually propagated by stolons in a nursery in Shasta County, California. Propagules were transplanted to a controlled breeding plot in Hillsborough County, Florida, where the variety was identified and selected for further evaluation. ‘Driscoll Bonaire’ was subsequently asexually propagated and underwent further testing in Hillsborough County, Florida for 5 years. This propagation and testing has demonstrated that the combination of traits disclosed herein which characterize the new variety are fixed and retained true to type through successive generations of asexual reproduction.

### SUMMARY OF THE INVENTION

The present invention relates to a new and distinct variety of strawberry named ‘Driscoll Bonaire.’ The variety is botanically identified as Fragaria×ananassa. The new variety is distinguished from other varieties by a number of characteristics as set forth in Tables 1-4.

### Comparison to Similar Varieties

The varieties which we believe to be similar to ‘Driscoll Bonaire’ from those known to us are ‘Driscoll Malibu’ (Patent Pending) and ‘Driscoll Osceola’ (U.S. Plant Pat. No. 15,752). There are several characteristics of the new variety that are different from, or not possessed by ‘Driscoll Malibu’ and ‘Driscoll Osceola’. For example, the new variety has a longer petiole length and wider petiole diameter, longer sepal length and greater sepal width, longer fruit length, greater berry weight and longer harvest interval.

‘Driscoll Bonaire’ is distinguished from its reference varieties, ‘Driscoll Malibu’ and ‘Driscoll Osceola’ as indicated in Tables 1-4.

The parents, ‘Driscoll Marathon’ (U.S. Plant Pat. No. 12,817) and ‘Driscoll Madeira’ (U.S. Plant Pat. No. 14,109), were not available for side by side comparison with ‘Driscoll Bonaire’. ‘Driscoll Bonaire’ differs from ‘Driscoll Marathon’ by being a less vigorous plant, having much greater flesh firmness and greatly improved skin strength. ‘Driscoll Bonaire’ differs from ‘Driscoll Madeira’ by having a much glossier skin and a brighter red color where ‘Driscoll Madeira’ was a darker red color.

### BRIEF DESCRIPTION OF THE DRAWINGS

The accompanying photographs show typical specimens of the new variety, including fruit, foliage and flowers, in color as nearly true as it is reasonably possible to make in color illustrations of these characteristics.

FIG. 1 shows the upper side of the leaves of the plant.

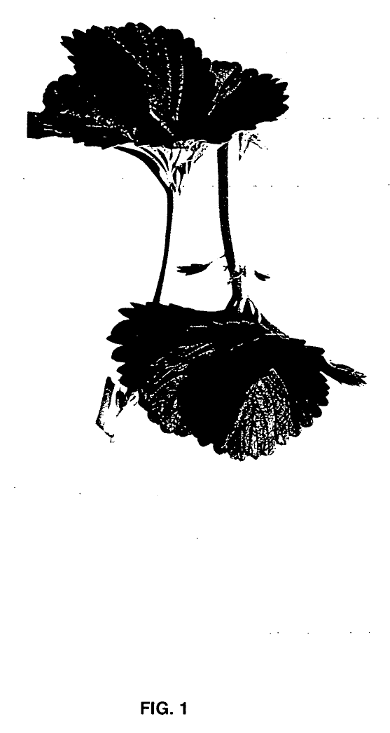

FIG. 2 shows the under side and upper side of the flowers.

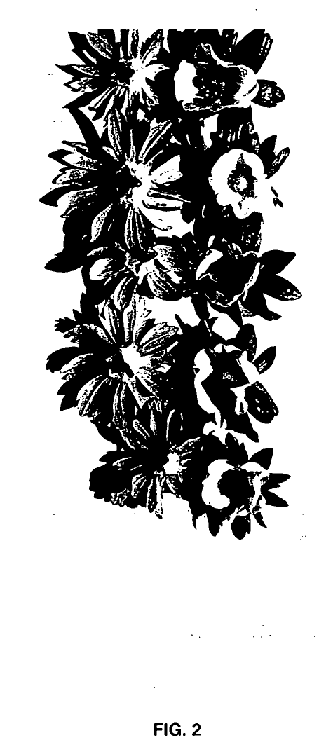

FIG. 3 shows a close-up of the fruit.

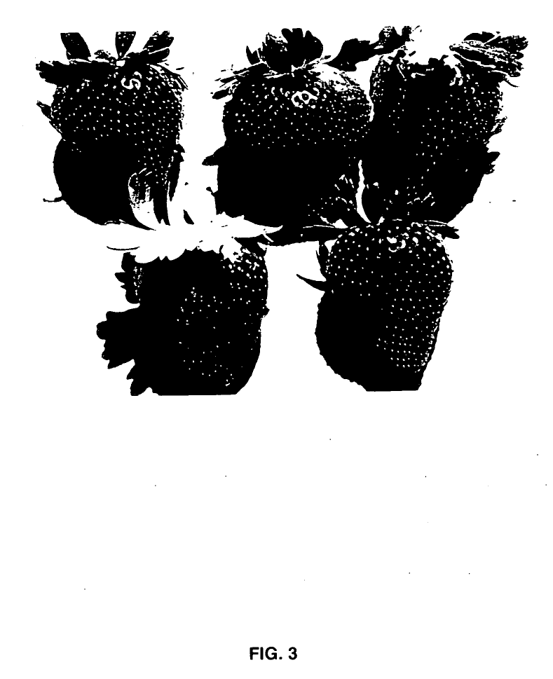

FIG. 4 shows the fruit in longitudinal cross-section.

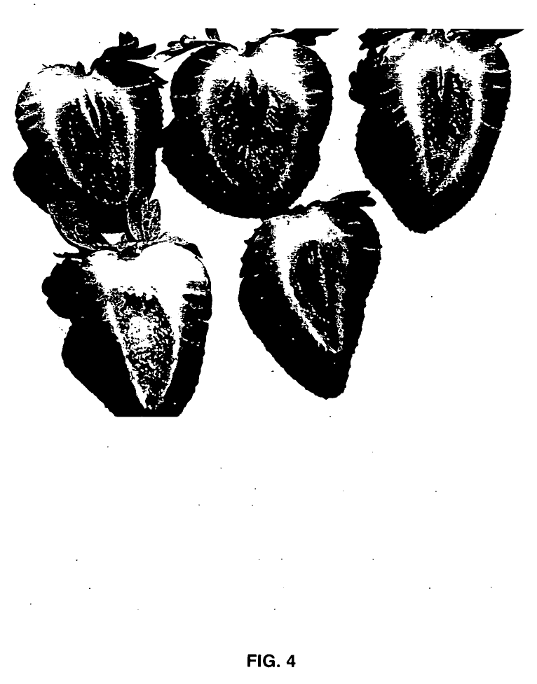

### DESCRIPTION OF THE NEW VARIETY

The following detailed description of the new variety is based upon observations taken of plants harvested from a nursery in McArthur, Calif. in September and October, 2004 and held in cold storage until planting in Hillsborough County, Florida in October, 2004. Plants were grown in raised beds of soil under conditions typical of commercial strawberry production in Hillsborough County, Florida. Fruits were harvested twice weekly from November, 2004 to April, 2005 for yield determination. Observations of ‘Driscoll Bonaire’, ‘Driscoll Malibu’, and ‘Driscoll Osceola’ were made in side by side comparison in January, 2005. Color designations, color descriptions, and other phenotypical descriptions may deviate from the stated values and descriptions depending upon variation in environmental, seasonal, climatic and cultural conditions. Colors are described and the most similar color designations are provided from The Royal Horticultural Society (RHS) Colour Chart.

### Propagation

The new variety is principally propagated by way of stolons. Although propagation by stolons is presently preferred, other known methods of propagating strawberry plants may be employed.

### Characteristics of the New Variety

Information on the new variety is presented in Tables 1, 2, 3 and 4. In the tables, the flowers described are secondary flowers except where indicated. The fruit described is the secondary fruit on four month old plants. Fruit and flower measurements are an average of both primary and secondary fruit and flowers.

Table 1 provides information on the plant and fruit characteristics of the new variety ‘Driscoll Bonaire’ compared with characteristics of ‘Driscoll Malibu’ and ‘Driscoll Osceola’. Table 2 provides additional information of the plant and fruit characteristics of the new variety ‘Driscoll Bonaire’ compared with characteristics of the varieties ‘Driscoll Malibu’ and ‘Driscoll Osceola’. Table 3 provides reactions of the new variety to stresses, pests and diseases compared with reactions of the varieties ‘Driscoll Malibu’ and ‘Driscoll Osceola’. Table 4 provides isozyme characteristics of the new variety as compared to that of the varieties ‘Driscoll Malibu’ and ‘Driscoll Osceola’.

TABLE 1
QUANTITATIVE COMPARISON OF ‘DRISCOLL BONAIRE,’
‘DRISCOLL MALIBU’ AND ‘DRISCOLL OSCEOLA’

| | | |
|---|---|---|
| ‘Driscoll | ‘Driscoll | ‘Driscoll |
| Bonaire’ | Malibu’ | Osceola’ |

Plant Characteristics

| | | | |
|---|---|---|---|
| Height of Plant (cm) | 9.2 | 11.4 | 8.3 |
| Spread of Plant (cm) | 32.4 | 33.9 | 31.2 |
| Number of Crowns | 3.7 | 4.9 | 3.8 |

Leaf Characteristics

| | | | |
|---|---|---|---|
| Terminal Leaflet | 7.8 | 6.6 | 6.7 |
| Length (cm) | | | |
| Terminal Leaflet Width | 7.9 | 6.6 | 8.1 |
| (cm) | | | |
| Terminal Leaflet | 0.98 | 0.99 | 0.83 |
| Length/Width Ratio | | | |
| # Teeth/Terminal | 25.7 | 22.5 | 23.8 |
| Leaflet | | | |
| Color of upper side | 132B | 141A | 135B |
| | medium green | light to | medium green |
| | | medium | |
| | | green | |
| Color of under side | 130B | 139C | 138B |
| | light green | light green | light green |
| Petiole Length (cm) | 7.7 | 10.3 | 8.0 |
| Petiole Diameter (mm) | 4.2 | 3.2 | 3.5 |
| Petiole Color | 142C | 141C | 140C |
| | green | green | green |
| Petiolule Length (mm) | 11.2 | 9.8 | 8.3 |
| Petiolule Diameter | 2.5 | 1.9 | 2.0 |
| (mm) | | | |
| Petiolule Color | 142C | 141C | 140C |
| | green | green | green |
| Bract Frequency | 92% | 83% | 8% |
| | typically paired | typically | typically single |
| | | paired | |
| Stipule Length (cm) | 3.07 | 3.46 | 3.53 |
| Stipule Width (cm) | 0.99 | 0.81 | 0.89 |

Stolon Characteristics

| | | | |
|---|---|---|---|
| Anthocyanin color | 185A | 58A | 59D |
| | greyed purple red | purple red | purple red |
| Diameter at bract (mm) | 3.09 | | |
| Avg. # of Daughter | 54 | 55 | 65 |
| plants (2003 Nursery) | | | |

Flower Characteristics

| | | | |
|---|---|---|---|
| Petal Length (cm) | 1.40 | 1.22 | 1.38 |
| Petal Width (cm) | 1.52 | 1.29 | 1.48 |
| Petal Length/Width | 0.92 | 0.95 | 0.93 |
| Ratio | | | |
| Flower Diameter (cm) | 2.94 | 2.57 | 2.83 |
| Calyx Diameter (cm) | 4.42 | 3.99 | 4.41 |
| Sepal Length (mm) | 18.3 | 15.6 | 16.9 |
| Sepal Width (cm) | 8.5 | 7.1 | 6.8 |
| Petal Color | 155C | 155C | 155C |
| | white | white | white |
| Receptical Cob | 1B | 1B | 1B |
| | yellow | yellow | yellow |
| Anther Color | 1A | 1A | 1A |
| | yellow | yellow | yellow |
| Fruiting Truss Leng | 13.5 | 14.4 | 11.8 |
| (cm) | | | |

Fruit Characteristics

| | | | |
|---|---|---|---|
| Fruit Length (cm) | 5.2 | 4.7 | 4.2 |
| Fruit Width (cm) | 4.1 | 3.7 | 3.8 |
| Fruit Length/Width | 1.25 | 1.25 | 1.13 |
| Ratio | | | |
| Average Berry Weight | 32.9 | 28.0 | 29.9 |
| (g) | | | |
| External Color | 46A | 46B | 46A |
| | red | red | red |
| Internal Color | 43A | 48C | 48D |
| | light red | orange red | orange red |
| Achene Coloration | 178B to 160B | 178C to | 178C to 161B |
| | | 160C | |
| | Greyed Orange to | Greyed | Greyed Orange |
| | Greyed Yellow | Orange to | to Greyed |
| | | Greyed | Yellow |
| | | Yellow | |
| Achenes per berry | 342 | 240 | 225 |
| Achene weight (g) | 0.00058 | 0.00062 | 0.00089 |
| 2004-2005 Marketable | 435 | 333 | 348 |
| Yield (g/plant) | | | |

TABLE 2
QUALITATIVE COMPARISON OF ‘DRISCOLL BONAIRE,’
‘DRISCOLL MALIBU’ AND ‘DRISCOLL OSCEOLA’

| | | |
|---|---|---|
| | ‘Driscoll | ‘Driscoll |
| ‘Driscoll Bonaire’ | Malibu’ | Osceola’ |

Plant

| | | | |
|---|---|---|---|
| Habit | flat globose | globose | flat globose |
| Canopy Density | medium | medium | open to |
| | | | medium |
| Vigor | medium | weak to | medium |
| | | medium | |

Leaf

| | | | |
|---|---|---|---|
| Shape in cross section | concave | slightly | concave to |
| | | concave to | slightly |
| | | flat | concave |
| Interveinal blistering | weak | medium | medium |
| Glossiness | medium | weak | medium |
| Number of leaflets | three only | three only | three only |
| Terminal leaflet margin | revolute | flat | flat |
| profile | | | |
| Terminal leaflet shape of | rounded | rounded | rounded |
| base | | | |
| Terminal leaflet shape of | obtuse | obtuse to | obtuse |
| teeth | | rounded | |
| Stipule pubescence | medium | medium | medium |
| Petiole pubescence | dense | dense | dense |
| Petiole pose of hairs | outwards | downwards | outwards to |
| | | | downwards |

Stolon

| | | | |
|---|---|---|---|
| Anthocyanin coloration | strong | medium to | medium to |
| | | strong | strong |
| Thickness | medium | medium to | medium to |
| | | thick | thick |
| Pubescence | medium | medium | dense |

Inflorescence

| | | | |
|---|---|---|---|
| Position relative to | level with | level to | level with |
| foliage | | above | to above |
| Diameter of calyx | larger | larger | larger |
| relative to corolla on | | | |
| secondary flowers | | | |
| Diameter of inner calyx | same size | larger | smaller to |
| relative to outer on | | | same size |
| secondary flowers | | | |
| Spacing of petals | overlapping | overlapping | overlapping |

Fruiting Truss

| | | | |
|---|---|---|---|
| Attitude at first picking | prostrate | prostrate | prostrate |

Fruit

| | | | |
|---|---|---|---|
| Predominant shape | conical | conical | cordate |
| Difference in shapes | slight | slight | slight |
| between primary and | | | |
| secondary fruits | | | |
| Band without achenes | very narrow | very | very |
| | | narrow | narrow |
| Unevenness of surface | weak | medium | weak |
| Evenness of color | slightly uneven | slightly | slightly |
| | | uneven | uneven |
| Glossiness | strong | strong | strong |
| Insertion of achenes | level with surface | below to | below |
| | | level with | surface |
| | | the surface | |
| Insertion of calyx | level | level | level |
| Pose of the calyx | reflexed | reflexed | spreading |
| segments | | | to reflexed |
| Size of calyx in relation | same size to | same size | same size |
| to fruit on secondary | larger | to larger | to larger |
| fruit | | | |
| Adherence of calyx | strong | strong | strong |
| Firmness of flesh | soft | soft to | soft to |
| | | medium | medium |
| Evenness of flesh color | slightly uneven | slightly | slightly |
| | | uneven | uneven |
| Distribution of flesh | marginal and | marginal | marginal |
| color | central | and central | and central |
| Hollow center size | small | small | medium |
| Sweetness | medium | medium | medium |
| Texture when tasted | fine | medium | fine |
| Acidity | medium | medium | medium |
| Time of First Flowering | late October | early | early |
| in 2003 | | November | November |
| Harvest Interval in 2003 | late November | early | early |
| | through early | December | December |
| | April | through | through |
| | | early April | early April |
| Type of Bearing | partially | partially | partially |
| | everbearing | everbearing | everbearing |

### Reaction to Stress, Pests, and Disease

TABLE 3
REACTIONS TO STRESS PESTS AND DISEASES FOR
‘DRISCOLL BONAIRE’, ‘DRISCOLL MALIBU’
AND ‘DRISCOLL OSCEOLA’

| | ‘Driscoll | ‘Driscoll | |
|---|---|---|---|
| | Bonaire’ | EI Capitan’ | ‘Driscoll Venice’ |
| Reaction to Pests | | | |
| Tetranychus urticae | susceptible | susceptible | susceptible |
| Lygus hesperus | susceptible | susceptible | susceptible |
| Reaction To | | | |
| Diseases | | | |
| Botrytis fruit rot | susceptible | susceptible | susceptible |
| Powdery mildew | moderately | moderately | moderately |
| | susceptible | susceptible | susceptible |
| Verticillium wilt | susceptible | highly susceptible | highly susceptible |
| Strawberry Mottle | susceptible | susceptible | susceptible |
| Virus | | | |
| Xanthomonas | moderately | moderately | moderately |
| fragariae | susceptible | susceptible | susceptible |

### Isozyme Analysis

In addition to the morphological description above, the new cultivar ‘Driscoll Bonaire’ has been analyzed to obtain an indication of its genetic makeup to provide further means for identifying the new variety and distinguishing it from other somewhat similar and/or related strawberry varieties. Specifically, leaf samples of ‘Driscoll Bonaire’, ‘Driscoll Malibu’ and ‘Driscoll Osceola’ were analyzed by electrophoresis for isozyme patterns of the enzymes phosphoglucoisomerase (“PGI”), leucine aminopeptidase (“LAP”) and phosphoglucomutase (“PGM”). See J. Amer. Soc. Hort. Sci. 106:684-687. Isozyme characterization of the three varieties is presented in Table 4, with the letters representing the banding patterns for each enzyme as designated in the above-identified article.

TABLE 4
ISOZYME ANALYSIS FOR ‘DRISCOLL BONAIRE,’
‘DRISCOLL MALIBU’ AND ‘DRISCOLL OSCEOLA’

| Locus | ‘Driscoll Bonaire’ | ‘Driscoll Malibu’ | ‘Driscoll Osceola’ |
|---|---|---|---|
| PGI | A1 | A1 | A1 |
| LAP | B3 | B3 | B3 |
| PGM | C2 | C4 | C4 |

What is claimed:

1. A new and distinct variety of strawberry plant, substantially as shown and described.
